(19) United States
(12) Patent Application Publication
(10) Pub. No.: US 2005/0124791 A1
(43) Pub. Date: Jun. 9, 2005

(54) Ion channel

(76) Inventors: Helge Volkel, Ulm (DE); Joachim E. Schultz, Ammerbuch (DE); Jurgen Linder, Tubingen (DE)

Correspondence Address: NATH & ASSOCIATES, 1030 15th STREET, NW, 6TH FLOOR, WASHINGTON, DC, 20005, US

(21) Appl. No.: 10/494,860

(22) PCT Filed: Nov. 8, 2002
(86) PCT No.: PCT/EP02/12508

(30) Foreign Application Priority Data
Nov. 8, 2001 (DE) 101 55 738.8
Nov. 8, 2001 (DE) 101 55 736.1

(51) Int. Cl. C12Q 1/68; C07H 21/04; C07K 14/705

(52) U.S. Cl. 530/350; 536/23.5; 435/69.1; 435/320.1; 435/325; 435/6

(57) ABSTRACT

The invention relates to a protein, which comprises an adenylate cyclase domain in addition to an ion channel domain. The ion channel domain is preferably a potassium ion channel domain, which is sensitive to voltage. In addition, the protein has a C-terminal protein-protein interaction domain. The invention also relates to nucleotide sequences, which code for proteins of this type.

The invention relates to a protein which has an ion channel domain, and to the corresponding nucleotide sequence and to the use of this nucleotide sequence and of the protein.

The cells of living organisms are distinguished inter alia by the ion ratios in the interior of cells and in the extracellular space having in each case very particular values. For example, in the interior of the cell, in contrast to the extracellular space, there is a low Na⁺ and Cl⁻ and a high K⁺ concentration. The various processes responsible for setting up and maintaining these particular ion ratios can be summed up by the term ion regulation.

So-called ion pumps are for one thing involved in ion regulation. These are transport proteins which transport ions through the membrane lipid layer by active, energy-consuming mechanisms. The principal supplier of energy for this process is ATP (adenosine triphosphate).

Ion channels form a second type of membrane-associated transport proteins. Ions are able to pass passively through the cell membrane through these channels. Various mechanisms are known for the opening and closing of these channels. For example, this control takes place by binding of an extracellular ligand (ligand-gated channels) or by voltage changes or by changing the membrane potential (voltage-gated channels). The ion concentration gradients regulated by the ion transport mechanisms mentioned are crucially important for organisms. For example, stimulus transmission in the nervous system takes place through BEAT AVAILABLE COPY electrical impulses which are caused by very particular ion currents. The membrane of nerve cells is distinguished by having a particular electrical polarization, called the membrane potential. Stimulation of the nerve cell causes an instantaneous reversal of the electrical polarization of the membrane, called the action potential. Voltage-controlled Na⁺ and K⁺ channels are responsible for this inter alia.

The unicellular ciliate Paramecium has been investigated in various ways as a model organism for nerve cells (neurons) of mammals, because it is able to form action potentials as a consequence of depolarization of the membrane (Satow, Y., Kung, C. (1974) Nature 247; 69-71). The electrophysiology of Paramecium has been investigated in detail. It was found from this that the membrane potential and the ion flux, especially the efflux of potassium ions out of the cell, is responsible for the formation of a signal molecule, cyclic 3′,5′-adenosine monophosphate (cAMP). A transient rise in cAMP was to be observed after hyperpolarization of the Paramecium cell. It was possible to prevent this effect specifically by blockers of potassium channels (Schultz, J. E., et al. (1992) Science 255; 600-603).

The enzyme adenylate cyclase (AC) is responsible for the formation of the intracellular signal molecule cAMP and catalyzes the conversion of adenosine triphosphate (ATP) into the cyclic adenosine monophosphate. Adenylate cyclases are found both in bacteria and in eukaryotic unicellular organisms (protozoa) and multicellular organisms (metazoa) (Barzu, O., Danchin, A., (1994) Prog. Nucleic Acid Res. Mol. Biol. 49; 241-283). At present there are suggested to be at least three classes of adenylate cyclases, which have no sequence similarities with one another. Representatives of adenylate cyclases of classes I and II are to be found in various bacteria. The adenylate cyclases of class III are the most widespread ACs and are detectable in many bacteria, in protozoa and in metazoa.

In mammals, the known membrane-bound ACs (class III) have a structure with two membrane domains each consisting of six transmembrane helices (Sunahara, R. K., et al. (1996) Annu. Rev. Pharmacol. Toxicol. 36; 461-480). The regulation known to date for mammalian ACs proceeds via hormones. After the hormones have bound to their particular receptors, the signals are transmitted to ACs via various routes. For example, G-proteins, protein kinases or Ca²⁺ ions are involved in this transmission.

The cAMP formation, mentioned at the outset, in the ciliate Paramecium appears not to be hormonally regulated. On the contrary, the regulation in this appears to take place via ion currents. It has been assumed that the adenylate cyclase activity and the ion conductance are brought about by a protein (Schultz, J. E., et al. (1992) Science 255; 600-603). However, it has not to date been possible to establish the identity of such a postulated enzyme.

A first attempt at identifying the AC from Paramecium started from the fact that this enzyme is closely related to known ACs of mammals. This approach led to the cloning and characterization of an enzyme from Parmecium which is similar to a mammalian AC. However, it emerged that this is a guanylate cyclase (GC) (Linder, J. U., et al. (1999) EMBO J. 18, 4222-4232). The existence of two similar GCs in Plasmodium was discovered by sequence comparisons and was subsequently confirmed biochemically (Carucci, D. J., et al. (2000) J. Biol. Chem. 275; 22147-22156). These results show that the enzymes which form cyclic nucleotides are closely related in ciliates such as Paramecium and in apicomplexa such as Plasmodium.

In the interim, various genome projects have been undertaken for Paramecium tetraurelia (Dessen, P., et al. (2001) Trends Genet. 17; 306-308) and for various Plasmodium species (Gardner M. J. et al. (1998) Science 282; 1126-1132; Bowman, S., et al. (1999) Nature 400; 532-538). In this connection, a partial sequence has been published for Paramecium and shows similarity to the catalytic domain of an AC. Substantially complete deciphering of the genome of Plasmodium falciparum has already been possible. However, an appropriate exon-intron structure which would form the basis for expression of the enzyme which is sought has not been discoverable.

The object of the invention is thus to identify a protein, or the corresponding nucleotide sequence, which is responsible for the close coupling of ion channel activity and adenylate cyclase activity, especially in Paramecium. It is intended to use this novel, previously undisclosed protein for identifying further corresponding enzymes from other organisms, especially from mammals. Identification of such ion channels is of particular interest because disturbances of ion channels are very important in a large number of disorders. Development of active substances which influence the activities of the novel proteins is therefore a further object of the invention.

The object is achieved by a protein as described in claim 1. Preferred embodiments of this protein and the corresponding nucleotide sequences are to be found in claims 2 to 19. The following claims 20 to 29 relate to various uses of the nucleotide sequences and proteins. Claim 30 is concerned with active substances of the invention. The wording of all the claims is hereby included in the description by reference.

For the purposes of the invention, the term “protein” is also usually intended to mean a peptide, which is, so to speak, part of the protein.

The protein of the invention is characterized in that it has an adenylate cyclase domain and an ion channel domain. The inventors have been able for the first time to show such an ion channel which simultaneously comprises an enzymatic activity, namely an adenylate cyclase activity.

The ion channel domain is preferably a potassium ion channel domain. However, other ion channels are also encompassed by the invention, for example sodium or calcium channels. The ion channel is advantageously voltage-controllable. Voltage-controlled ion channels play a very important part in many processes in the organism. For example, voltage-controlled ion channels are involved in stimulus transmission, especially in the formation of action potentials.

In a preferred embodiment of the protein of the invention, the ion channel domain has six transmembrane helices and a pore loop. The fourth transmembrane helix for example is the voltage-sensitive helix. The pore loop is preferably disposed after the sixth transmembrane helix and preferably projects from the intracellular side into the cell membrane. This is a crucial difference from known ion channels, where the pore loop is usually located between the fifth and sixth transmembrane helix and, in this case, extends from the extracellular space into the cell membrane.

The protein of the invention is further characterized by a protein-protein interaction domain. This is preferably a so-called tetratricopeptide repeat-like (TPR) domain. A domain of this type is important for the ability of the protein of the invention to function. Such domains have already been described in connection with other enzymes. However, combination with an adenylate cyclase, as in the protein of the invention, has not previously been disclosed.

In a preferred embodiment of the invention, the protein-protein interaction domain is disposed C-terminally in the complete protein. The ion channel domain of the protein of the invention is, in a preferred embodiment, located in the N-terminal region of the complete protein.

In a preferred embodiment of the invention, the functional unit of the protein is formed by a tetramer. In this case, four protein chains in each case form a pore, that is to say the ion channel, and in each case two protein chains form a catalytic domain of the adenylate cyclase, so that the ion channel-AC tetramer has in each case two AC dimers and one pore tetramer.

In a particularly preferred embodiment of the invention, the protein of the invention is characterized in that it is encoded at least in part by a nucleotide sequence which is at least 65%, in particular at least 70%, identical to a nucleotide sequence as shown in SEQ ID NO 1 and/or SEQ ID NO 2, or parts thereof.

SEQ ID NO 1 shows the cDNA sequence which codes for the protein of the invention from Paramecium tetraurelia. The open reading frame starts at nucleotide 2. The stop codon is located at nucleotide 2597. The different codon usage of Paramecium means that the triplets TAA and TAG code for glutamine. SEQ ID NO 2 shows the Plasmodium falciparum cDNA sequence coding for the protein of the invention from this organism. In this case, the open reading frame starts at nucleotide 1. The stop codon is located at nucleotide 2655.

The invention also encompasses proteins and peptides which are characterized in that they are encoded at least in part by a nucleotide sequence as shown in SEQ ID NO 1 or parts thereof and/or SEQ ID NO 2 or parts thereof. These therefore substantially comprise the corresponding protein from Paramecium tetraurelia or from Plasmodium falciparum or parts of these proteins, such as, for example, the part having the adenylate cyclase activity or having the ion channel activity. The invention additionally encompasses proteins and peptides which are encoded at least in part by a nucleotide sequence as shown in SEQ ID NO 5 and/or No 6.

The proteins of the invention can, for example, be isolated from an organism and also purified. However, it is particularly preferred for the protein to be expressed in an experimental system. Suitable for this are essentially all expression methods familiar to the skied worker. Expression in a heterologous system is particularly advantageous, for example expression in insect cells such as, for example, Sf9 cells using the baculovirus technique. It may in this case be advantageous to express in each case only particular parts of the protein of the invention, such as, for example, the adenylate cyclase catalytic domain.

The invention further encompasses nucleotide sequences or parts thereof which are at least 65%, in particular at least 70%, identical to a nucleotide sequence as shown in SEQ ID NO 1 and/or SEQ ID NO 2. The invention additionally encompasses the nucleotide sequence as shown in SEQ ID NO 1 or parts thereof and the nucleotide sequence as shown in SEQ ID NO 2 or parts thereof. The invention moreover encompasses the nucleotide sequence as shown in SEQ NO 5 or parts thereof and the nucleotide sequence as shown in SEQ ID No 6 or parts thereof. These nucleotide sequences are advantageously characterized in that they code for a protein having an adenylate cyclase domain and/or an ion channel domain, in particular a potassium ion channel domain. These nucleotide sequences may be in isolated form. They may also, for adaptation to the particular purpose of use, be incorporated in a vector such as, for example, an expression vector. These sequences may additionally be combined with other sequences.

The invention further encompasses proteins or peptides which are characterized in that they are encoded at least in part by a nucleotide sequence which is at least 65%, in particular at least 70%, identical to a nucleotide sequence as shown in SEQ ID NO 1 and/or SEQ ID NO 2, or parts thereof.

Additionally encompassed are proteins and peptides which are encoded at least in part by a nucleotide sequence as shown in SEQ ID NO 1 or parts thereof and/or SEQ ID NO 2 or parts thereof. These therefore substantially comprise the corresponding protein from Paramecium tetraurelia or from Plasmodium falciparum or parts of these proteins, such as, for example, the part having the adenylate cyclase activity or having the ion channel activity. The invention further encompasses proteins and peptides which are encoded at least in part by a nucleotide sequence as shown in SEQ ID NO 5 and/or NO 6.

The protein of the invention is characterized in that it has an adenylate cyclase domain and/or an ion channel domain. The inventors have been able to identify for the first time a protein which has an ion channel and simultaneously an enzymatic activity, namely an adenylate cyclase activity.

Concerning further properties of the proteins and peptides of the invention, reference is made to the above description.

The nucleotide sequences of the invention described hereinbefore are further characterized in that they code for a peptide or protein as described herein.

The invention further encompasses the use of said nucleotide sequences for identifying similar nucleotide sequences, in particular for identifying similar nucleotide sequences from mammalian cells.

There is a noticeable similarity between the sequence which codes for the adenylate cyclase catalytic domain in the protein of the invention and the sequence for the catalytic domain of the soluble adenylate cyclase from rat testis. This fact and the occurrence of the proteins of the invention in the ciliate Paramecium and in the parasite Plasmodium implies the existence of such proteins in a large number of organisms including mammals.

These homologous proteins can be identified with the aid of the nucleotide sequences of the invention.

Preferably bioinformatic and/or immunological methods are employed for this purpose. For example, cross-reaction of antibodies against the protein of the invention from Paramecium and/or Plasmodium can be tested in other organisms in order to identify in this way the related proteins and eventually also the respective nucleotide sequences. Antibodies can be produced by using for example the complete protein of the invention or only particular parts thereof. Suitable epitopes are in particular the N-terminus of the complete protein or the adenylate cyclase catalytic domain. Appropriate methods for producing the antibodies and for identifying the homologous sequences and proteins with the aid of the antibodies or with the aid of bioinformatic methods are familiar to the person skilled in this art.

In addition, the invention encompasses nucleotide sequences which have been identified in accordance with the use just described. These sequences are therefore ones coding for proteins which are similar to the previously described proteins of the invention or are related thereto. The invention also encompasses in this connection the corresponding peptides or proteins encoded by these identified nucleotide sequences. Proteins of this type from mammals are particularly preferred in this connection.

The invention additionally encompasses the use of the nucleotide sequences described above or of the nucleotide sequences which have been identified as just described, and of the peptides or proteins encoded thereby for developing active substances. For this use, the corresponding sequences are advantageously expressed in a system which is familiar to the skilled worker and thus made available for experimental approaches. Particularly suitable for this purpose are heterologous expression systems such as, for example, expression in insect cells using the baculovirus technique. It is possible by investigating the interaction of the nucleotide sequences of the invention or of the corresponding peptides or proteins with potential active substances to develop and/or identify substances which influence the activity of the proteins of the invention, especially in a mammal. It is thus possible for example for the ion channel activity to be activated, inhibited, or modulated in another way. A further possibility is also to increase, inhibit and/or modulate the adenylate cyclase activity of the protein of the invention. The active substance may also in addition act on the protein-protein interaction domain. Said effects of the active substance may be brought about in each case singly or else in combination by the active substance. Whether activation, inhibition or other modulation is advantageous in each case depends on the particular application.

The nucleotide sequences employed for this use are preferably derived from the organism Plasmodium spec. or substantially correspond to the nucleotide sequence from this organism. Various representatives of the genus Plasmodium are causative agents of malaria. Each year more then one million people die from this disease which is currently distributed world-wide in the tropics and, in some cases, also in the subtropics. The pathogen of the most severe form of malaria, tropical malaria, is Plasmodium falciparum. The proteins of the invention or the corresponding nucleic acids represent a suitable starting point for the development of active substances for the treatment of malaria. Suitable active substances are in principle all substances evident to the person skilled in this art, such as, for example, peptides, proteins, nucleic acids, e.g. antisense sequences, or inorganic substances. The active substances which have been developed according to the invention and which are intended in particular for the treatment of malaria are likewise encompassed by the invention.

In a further preferred embodiment, the active substances developed according to the invention are intended for the treatment of cardiovascular disorders and/or epilepsy. Potassium channels are known to play a particularly outstanding part in these diseases, so that active substances which act on such channels are of very particular pharmacological interest. The corresponding active substances can, of course, also be employed for the treatment of other diseases which are associated with dysfunctions of ion channels and/or adenylate cyclases, and in particular with dysfunctions of the proteins of the invention, or which can be beneficially influenced in their progress by influencing these proteins. A further, particularly preferred, area of use of the active substances of the invention are disorders of the sensory organs such as, for example, of the eye or of the inner ear. The active substances developed according to the invention are likewise encompassed by the invention.

The described expression systems of the proteins of the invention are also suitable for identifying proteins associated with and/or functionally linked to the proteins of the invention. This application is of interest in particular for research. It is additionally possible with results achieved thereby to draw further starting points for the development of active substances for the treatment of diseases.

Further features of the invention are evident from the following description of the examples in conjunction with the figures and the dependent claims. The various features can in each case be implemented singly or in combination with one another.

The figures show:

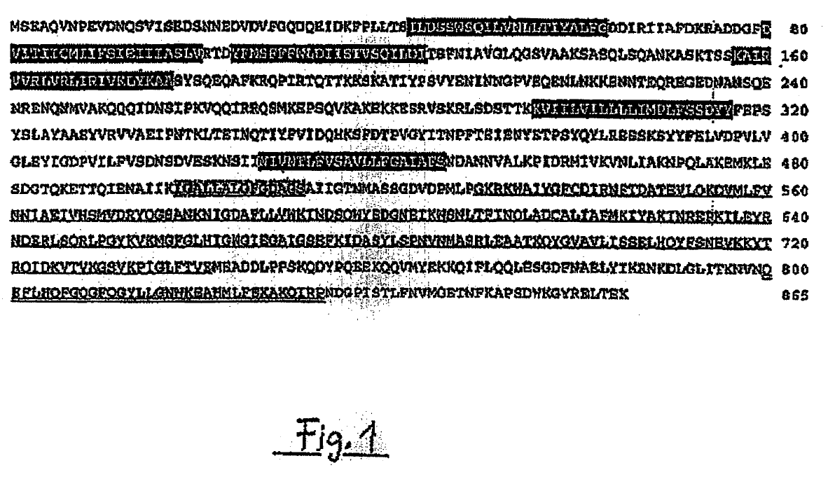

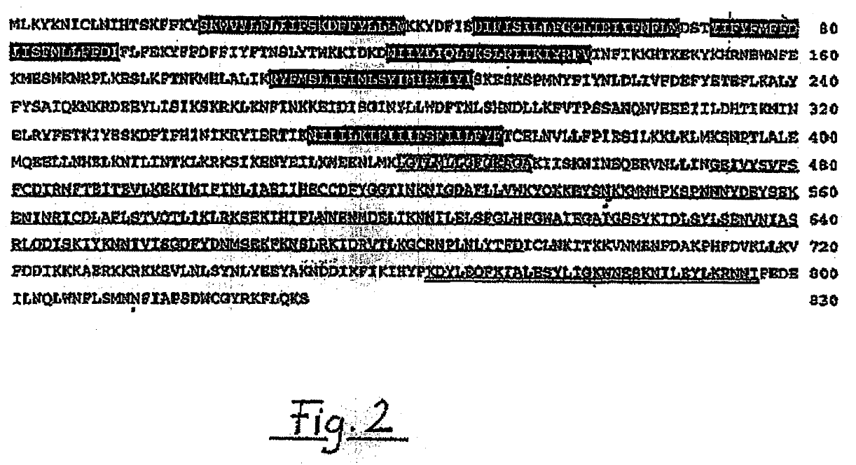

FIG. 1 the amino acid sequence of the protein of the invention from Paramecium tetraurelia. The six transmembrane helices have a black background and the pore loop a grey background. The catalytic domain is underlined, and the TPR-like domain is doubly underlined.

FIG. 2 the amino acid sequence of the protein of the invention from Plasmodium falciparum. The six transmembrane helices have a black background and the pore loop a grey background. The catalytic domain is underlined, and the TPR-like domain is doubly underlined.

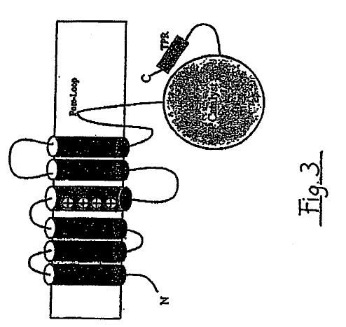

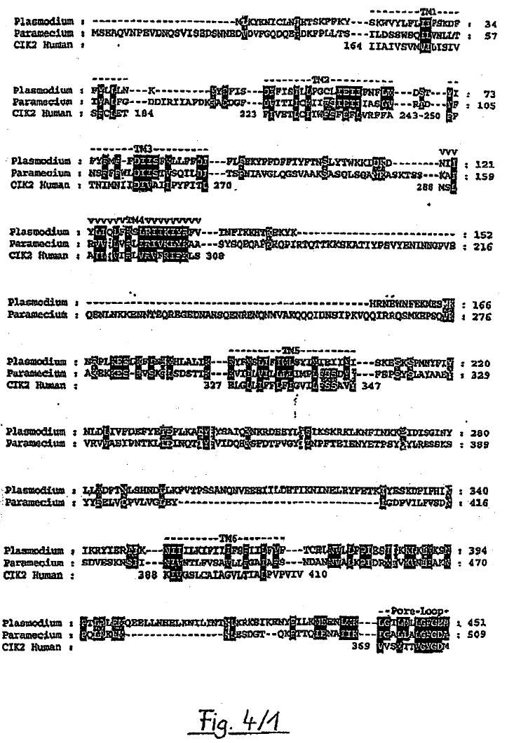

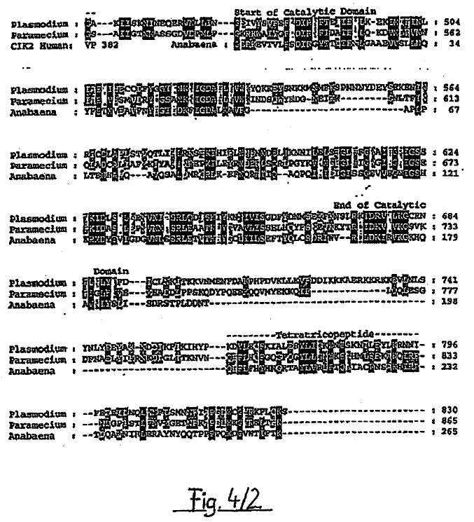

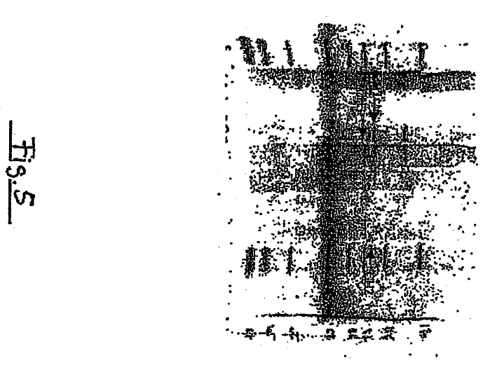

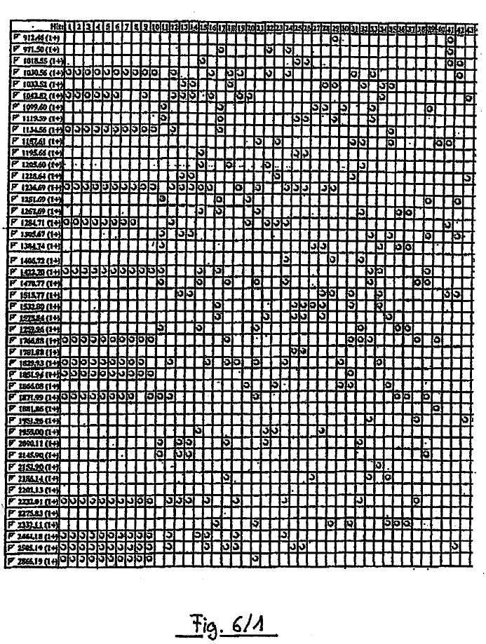

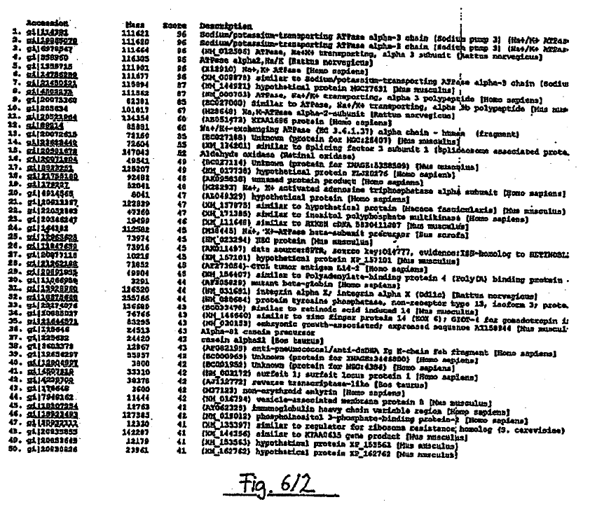

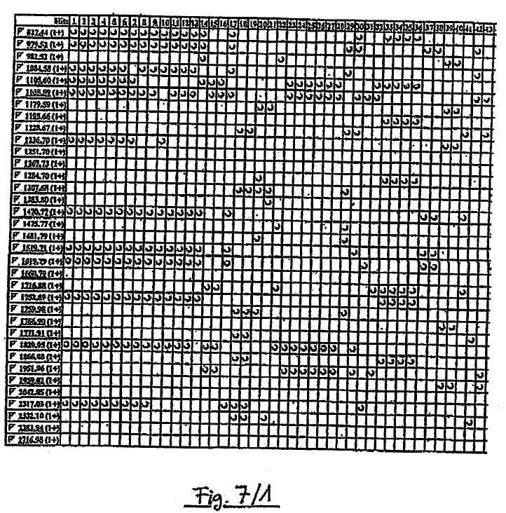

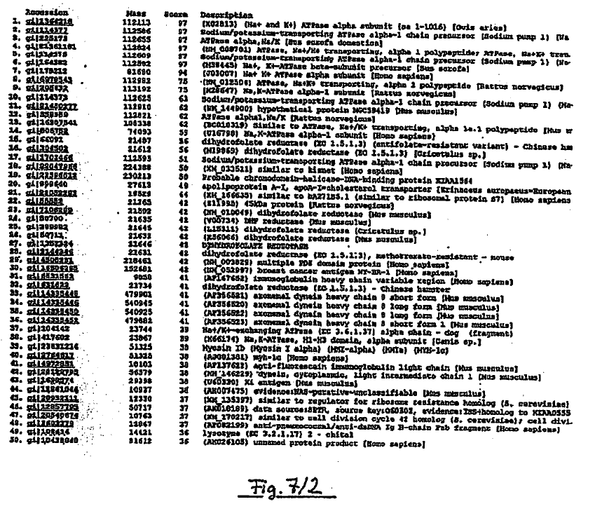

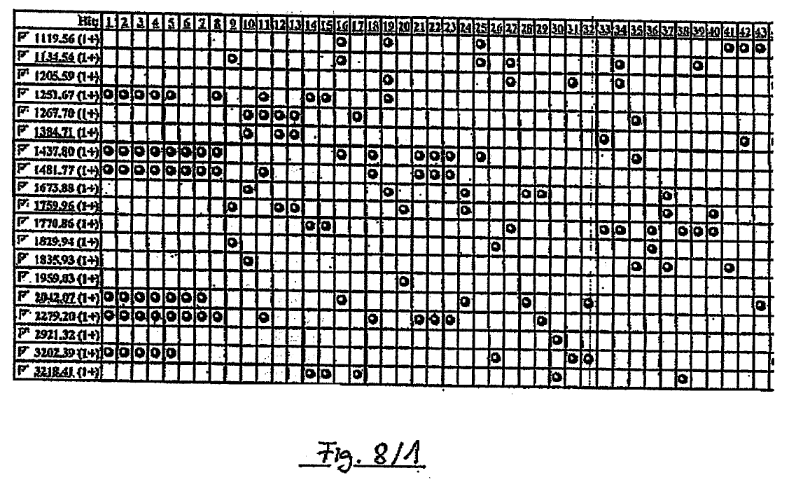

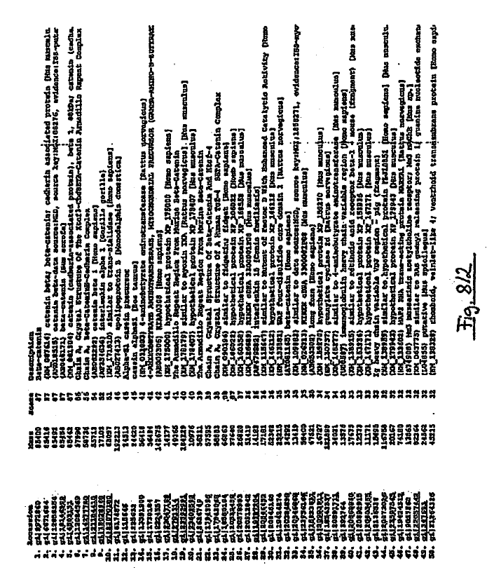

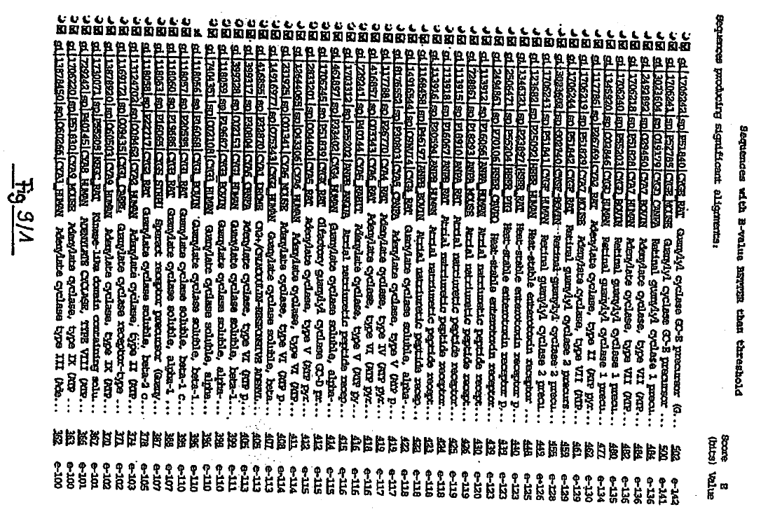

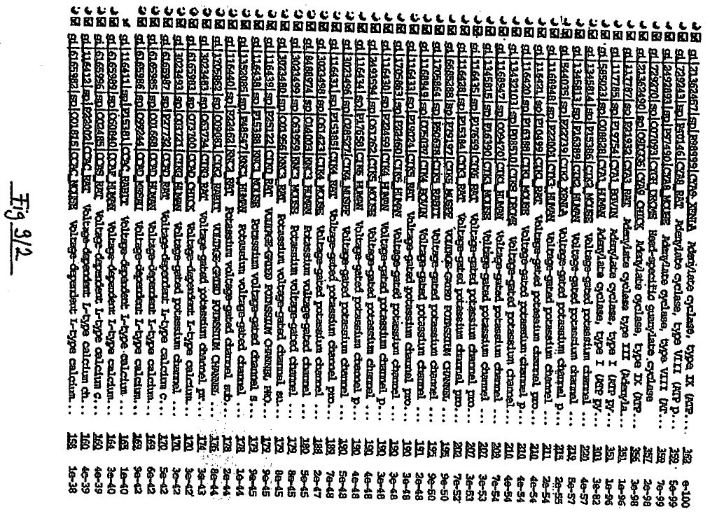

FIG. 3 the calculated topology of the protein of the invention. The cell membrane is depicted by thin lines. The transmembrane helices are symbolized by columns. The fourth transmembrane helix forms a voltage sensor and has a high positive charge. The classical pore loop of ion channels is located C-terminally from the sixth transmembrane helix.

FIG. 4 a comparison of the proteins of the invention from Paramecium and Plasmodium in combination with the individual transmembrane helices and the pore of the human inward rectifier C1K2 (GenBank accession No. L02752) and the C-terminal 265 amino acids of the adenylate cyclase CyaB1 from Anabaena (GenBank accession No. D89623). Conserved regions between these three sequences and some significant conserved regions between the proteins from Paramecium and Plasmodium in the channel domains have black backgrounds. Conserved regions between two sequences have grey backgrounds.

FIG. 5 silver-stained SDS polyacrylamide gel of the purified adenylate cyclase from the outer segment of the bovine retina. The gel shows markers in each of the outer lanes, between which are various fractions of the purified enzyme. CH1, CH2 and CH3 designate the bands to which the enzymatic activity is to be assigned.

FIG. 6 results of comparison of the MALDI-MS hits with the Paramecium sequence of the invention for the band CH1.

FIG. 7 results of comparison of the MALDI-MS hits with the Paramecium sequence of the invention f or the band CH2.

FIG. 8 results of comparison of the MALDI-MS hits with the Paramecium sequence of the invention for the band CH3.

FIG. 9 results of comparison of the Paramecium protein sequence of the invention with translated gene database information.

### SUMMARY OF THE SEQUENCE LISTING

SEQ ID NO 1 nucleotide sequence of the invention from Paramecium tetraurelia

SEQ ID NO 2 nucleotide sequence of the invention from Plasmodium falciparum

SEQ ID NO 3 amino acid sequence of the invention from Paramecium tetraurelia

SEQ ID NO 4 amino acid sequence of the invention from Plasmodium falciparum

SEQ ID NO 5 artificial expression cassette of the V; adenylate cyclase from Paramecium tetraurelia. The open reading frame is preceded by a Kozak sequence. The open reading frame is flanked at the 5′ end by the EheI restriction site and at the 3′ end by NotI.

SEQ ID NO 6 artificial expression cassette of the adenylate cyclase from Plasmodium falciparum. The open reading frame is preceded by a Kozak sequence. The open reading frame is flanked at the 5′ end by the HpaI restriction site and at the 3′ end by NotI.

### EXAMPLES

### Example 1

Degenerate primers for a polymerase chain reaction (PCR) were designed on the basis of a sequence on chromosome 14 of Plasmodium falciparum, which shows significant similarity with class III adenylate cyclases at the protein level. A PCR with total DNA from Plasmodium tetraurelia 51s revealed a DNA fragment which codes for a protein sequence which has great similarity with the catalytic region of class III adenylate cyclases. Subsequent screening of cDNA and total DNA libraries revealed the complete sequence of the AC from Paramecium tetraurelia (SEQ ID NO 1). The protein (SEQ ID NO 3, FIG. 1) encoded thereby is calculated to have a mass of 98 kDA. Analysis of the amino acid sequence shows three principal domains: an N-terminal ion channel domain (amino acids 1-514) which is connected to a catalytic adenylate cyclase domain (amino acids 530-741) and to a C-terminal tetratricopeptide repeat-like (TPR) domain (amino acids 800-833). This topology of the enzyme is shown in FIG. 3.

A subsequent PCR with total DNA from Paramecium revealed various other fragments of possible adenylate cyclases. This suggests that the cloned gene is part of a large family of AC isoforms in Paramecium.

Starting from the amimo acid sequence of the Paramecium protein, the data available from the Plasmodium falciparum genome project were examined. DNA regions which surround the catalytic region of the putative Plasmodium AC and which show significant similarities with Paramecium AC at the protein level were found thereby. Subsequently, a reverse transcription (RT) PCR was carried out employing specific primers according to the provisional sequence data for chromosome 14. A total of 23 introns was identified in this way, and these give the cDNA sequence shown in SEQ ID NO 2. The corresponding protein (SEQ ID NO 4, FIG. 2) has an identical topology to the protein from Paramecium with an ion channel domain, a catalytic AC domain and a TPR domain.

Comparison of the Paramecium protein with that from Plasmodium (FIG. 4) and with known ion channels and adenylate cyclases shows various structural features of these enzymes: the ion channel domain comprises six putative transmembrane helices. The fourth helix corresponds exactly with the classical voltage sensor of the voltage-sensitive ion channels. The helix consists of a very highly positively charged amphipathic peptide in which polar residues are arranged in the same way as in voltage sensors of ion channels (FIG. 4). The pore loop of classical ion channels is located between the fifth and sixth transmembrane helices. In contrast to this, the corresponding sequence in the ion channel of the protozoal proteins of the invention is located downstream from the sixth transmembrane helix, near to the N terminus of the catalytic AC domain (FIG. 4). The pore loop projects from the cytosolic side into the cell membrane. This is comparable with the potassium channel of the type of glutamate receptor from Synechocystis species (Chen, G. Q., et al. (1999) Nature 402; 817-821).

The catalytic AC domain shows the greatest similarity with bacterial class III adenylate cyclases, for example from Anabaena, Rhizobium and Treponema. The similarity with other adenylate cyclases from protozoa and metazoa is distinctly less. One exception to this is the soluble adenylate cyclase from rat testis, which displays distinct similarities with the catalytic AC domain of the protein of the invention. This type of class III adenylate cyclases thus appears to be distributed between bacteria, protozoa and also metazoa.

The TPR domain at the C terminus of the protein of the invention exists not only in this protein having adenylate cyclase activity from Paramecium and Plasmodium but also in the adenylate cyclase CyaB1 (FIG. 4) and CyaB2 from Anabaena spec. and in the adenylate cyclase ACr from Dictyostelium discoideum.

In order to confirm the enzymatic activity of the AC domains of the Paramecium and Plasmodium proteins of the invention, the enzymes were heterologously expressed in various cell types. One problem with this is that the ciliate Paramecium uses an alternative genetic code, i.e. the universal TAA/TAG stop codons code for glutamine. Direct heterologous expression of Paramecium genes is therefore impossible. In addition, the cDNA of the Plasmodium AC domain has an extremely high A/T content (80%). This prevents efficient expression in established systems. In order to bypass these problems, artificial genes of the Paramecium AC and Plasmodium AC which use the mammalian codon usage were created (SEQ ID NO 5, SEQ ID NO 6).

Expression of the catalytic domain of the Paramecium AC in E. coli led to the production of large amounts of inclusion bodies. However, no AC activity was attained. A denaturing purification of the expressed protein yielded sufficient material to generate antibodies against the enzyme.

In contrast to this, expression of the catalytic domain of the Plasmodium AC in E. coli was very inefficient. Insect cells (Sf9 cells) were therefore employed as expression system, using the baculovirus technique. Expression of the plasmodium AC was successful. An AC activity was attained using a minimal construct having the amino acids 472-830, which encompass the catalytic AC domain and the TPR domain. An N-terminal hexahistidine tag permitted partial purification of the active enzyme by metal affinity chromatography. A larger construct which comprised amino acids 457-830 was likewise active and could also be purified. This construct additionally comprised the connection between the catalytic AC domain and the ion channel. It was thus possible to show that the Plasmodium adenylate cyclase is able to catalyze the formation of cAMP from ATP. This enzymatic activity of the protein of the invention functions independently, and this catalytic activity is located in the C-terminal part of the complete protein.

### Example 2

The Adenylate Cyclase from the Outer Segment of the Bovine Retina Having Homology with the Paramecium Ion Channel-Adenylate Cyclase

A new type of adenylyl cyclase was purified in 1992 (Schultz J E, Klumpp S, Benz R, Schurhoff-Goeters W J, Schmid A (1992) Regulation of adenylyl cyclase from Paramecium by an intrinsic potassium conductance. Science 1992 255; 600-603) from the cilia of Paramecium, which are very closely related physiologically and developmental biologically to the rods and cones of the mammalian retina. The protein which had been concentrated 10 000-fold and was 99% pure showed both enzymatic adenylyl cyclase activity and ion channel activity in black lipid bilayers. However, the sequence was unknown at that time. It has now been possible according to the invention to identify by homology cloning an adenylyl cyclase having a potassium channel pore from Paramecium, inter alia.

To investigate the question of whether a comparable protein also exists in multicellular organisms, especially in mammals, the protein sequence of the Paramecium AC (AC_PARA) was used as query to screen the translated gene database (NCBI, as at October 2002). The NCBI PSI/PHI-Blast algorithm was used, employing the PHI pattern [WIFV]FxxE. After 22 iterations, unambiguous identities with potassium channels in the N-terminal segment and with adenylyl/guanylyl cyclases were found (FIG. 9).

In 1994, an adenylate cyclase having similar physiological properties to the Paramecium adenylate cyclase was purified from membranes of the outer segments of the retina (bovine): cAMP formation activity and ion channel activity (thesis, Tübingen University, Susanne Otto (1994) Reinigung und Charakterisierung einer Adenylatcyclase der Retina).
Briefly: the enzyme was solubilized from retinal membranes with Lubrol PX (2%) in a yield of 71% and with 7.8-fold concentration. Purification took place in seven column chromatography steps. A 2-3-fold concentration was achieved with the DEAE-Trisacryl anion exchanger. A further 1.4-fold concentration was achieved with subsequent hydrophobic interaction chromatography on phenyl-Sepharose. This was followed by two affinity chromatography steps: lentil lectin-Sepharose (2.5-fold concentration) and ADP-agarose (3-fold concentration). Concentration by Mono-Q ion exchange chromatography (factor 1.2) was followed by gel filtration on Superdex 200 (concentration factor 2). Finally, a further concentration by a factor of 50 was achieved on ATP-agarose. The overall concentration of the AC starting from retina membranes was 15 000-fold. Only four protein bands were now unambiguously identifiable (Blum silver stain) by SDS-PAGE (FIG. 5). The enzymic activity is to be assigned to the 3 protein bands (CH1, CH2, CH3) in the region of 97 kDA, a comparable molecular weight to that of the Paramecium/Plasmodium AC. Neither the membrane-associated nor the purified soluble enzyme can be stimulated by GTP, GTPyS, GMPPhT and NaF. Ca²⁺/CaM activates the membrane-bound AC to twice the activity, but the purified enzyme is not Ca²⁺/CaM-sensitive. 100 pM forskolin stimulates the enzyme in the starting material up to three-fold, and the highly purified enzyme is activated up to seven-fold. The K_{M} values for the purified AC were 25 pM MnATP and 65 pM MgATP. It differed slightly in the pH optimum (pH 8) and in the molar energy of activation (72.6 kJ/mol) from membrane-bound and soluble enzyme. The purified AC was incorporated into a lipid double membrane. A single-channel conductance of about 100 pS was measured with 1M KCl on both sides of the membrane and with a membrane voltage of 50 mV. During the purification there was concentration both of AC activity and of ion channel activity measured in a lipid double membrane.

At that time it was not possible to obtain information about the sequence of the retina outer segment AC with channel activity either by cDNA homology cloning or by protein chemical analyses. For the identification according to the invention of the 3 bands in the region of 97 kDa (CH1, CH2, CH3), the silver-stained bands were isolated by standard proteomics methods (in-gel digestion).
Briefly: In Gel Digestion Protocol for Silver Stained Gels, Reagents: H₂O: Nanopure water, Acetonitrile: HPLC grade, Acetic Acid: JT Baker UltrexII Ultrapure, or equivalent, Formic Acid: EM Science ACS 88%, or equivalent, 100 mM bicarbonate: 0.2 g ammonium bicarbonate+20 mL H₂O, 50 mM bicarbonate: 3 mL 100 mM bicarbonate+3 mL H₂O, Extraction buffer: 50% Acetonitrile/5% Formic Acid, 10 mL Acetonitrile+9 mL H₂O+1 mL Formic acid, Procedure: Excised protein spots must be destained before mass spectrometry: Prepare: 30 mM (10 mg/mL) K₃Fe(CN)₆ and 100 mM (25 mg/mL) Na₂S₂O₃.5H₂O as stock solutions in water. The working solution is made freshly as a 1:1 mixture. Excise spots into ACN, remove SN. Cover each spot with 30-50 μL of working solution and vortex occasionally until brownish color disappears. Rinse several times with water. Cover spot with 200 mm NH₄HCO₃ for 20 min. Remove supernatant and cut gel into small pieces. Dehydrate gel pieces in 200 μL Acetonitrile. Remove Acetonitrile. Dry gel pieces in speed vac for 2-3 minutes. Wash with; 100 mM ammonium bicarbonate. Remove ammonium bicarbonate. Dehydrate in 200 μL Acetonitrile for 5 minutes. Remove Acetonitrile. Rehydrate in 200 μL of 100 mM ammonium bicarbonate for 5 minutes. Remove ammonium bicarbonate. Dehydrate in 200 μL Acetonitrile for 5 minutes. Remove Acetonitrile. Dry gel pieces in speed vac for 2-3 minutes. Prepare trypsin. 20 kg Promega modified trypsin+1000 uL ice cold 50 mM ammonium bicarbonate (20 ng/uL trypsin). Add 50 μL of trypsin to each gel piece. Rehydrate the gel on ice for 10 minutes. Microfuge and remove excess trypsin. Add 20 μL of 50 mM ammonium bicarbonate. Vortex and microfuge briefly. Digest overnight at 37° C. Cover tubes with kimwipe to avoid any dust contamination. Microfuge samples briefly. Add 30 μL 50% Acetonitrile/5% Formic acid, vortex for 10 minutes, microfuge. Collect supernatant in 0.5 ml eppendorf tube. Add 30 μL 50% Acetonitrile/5% Formic Acid to the gel piece. Vortex for 10 minutes, microfuge. Combine supernatant in eppendorf tube. Dry sample before Mass-spectrometric analysis.

Subsequently, the samples after tryptic digestion were analyzed by MALDI-TOF mass spectrometry or quadropole ESI mass spectrometry and, in this way, the retina AC bands were identified.
Briefly: Protocol for protein ID with MALDI-TOF MS. Chemicals and solvents used acetonitrile (UVAsol or equivalent) trifluoroacetic acid (TFA) (98%, highest obtainable degree of purity) α-cyano-4-hydroxycinnamic acid (HCCA or ACCA) of the highest obtainable degree of purity ZipTips, μ-C18, Millipore. The digested protein from the gel electrophoresis is in the form of, a lyophilizate and is dissolved in 5 to 20 μl of ACN/0.5% TFA (1:9) (depending on the “thickness” of the gel spot) and alternately vortexed for 3×20 sec and held in an ultrasonic bath. The Eppis are briefly centrifuged in order to collect the solution on the bottom. 1 μl of the analyte solution is applied to a steel target, initially without ZipTips, and mixed with 1 μl of a saturated HCCA solution (HCCA in ACN/0.1% TFA, 7:3). The spot should dry in air (possibly heat gently). The mass spectrometer is calibrated at the start of the measurements. This takes place using standard peptides covering a range of mass from m/z 1000 to m/z 3000. The dry preparation is measured in a mass spectrometer, initially recording only a peptide mass fingerprint (PMF) (MS only). For this purpose, typically 1000-5000 laser shots are totaled. The spectrum is if possible recalibrated internally (e.g. with trypsin signals) in order to be able to achieve accuracies of ±5 ppm in mass. If crystallization of the analyte spot on the target is poor or absent (gelatinous), or if no signals other than matrix signals can be detected, the analyte solution probably contains too much salt and/or too low an analyte concentration. In this case, the sample is “ziptipped”. This leads to an increase in the concentration of some of the tryptic fragments present in the solution and thus to possible exceeding of the limit of detection. At the same time, the sample is desalted. Good planning is necessary on use of ZipTips withthin samples because the solution usually no longer contains detectable amounts of tryptic peptides after the procedure. A peak list is generated from the resulting spectrum and is automatically transferred into a database search program. Mostly used: Mascot (www.matrixscience.com) and Prospector (prospector.ucsf.edu). The search result can be clearly verified and limited by a large number of adjustable parameters. Parameters typically used, (example): Database searched: NCBInr.user
Digest used: Trypsin, Max. # Missed Cleavages: 2, Peptide N terminus: Hydrogen, Peptide C terminus: Free Acid, Cysteine Modification: unmodified, Instrument Name: TOF-TOF, Sample ID (comment): what is actual?, Minimum Matches: 4, Sort Type: Score Sort, Considered modifications:|Peptide N-terminal Gln to pyroGlu|Oxidation of M|Protein N-terminus Acetylated|Min Parent Ion Matches: 1, MOWSE On: 1. The following points are used to analyze the hit list: only hits with a minimum of 4 “matching” peptides are to be taken into account. The score (a value which is calculated in various ways, and is produced in a complicated manner, for the reliability of a hit) is usually quite large for the first hit in the list. When this value falls to about 1/10, hits below this value should be ignored. The deviations in mass of the peptides taken into account (differences between “calculated” and “observed”) must be with low tolerances in analogy to the deviation in mass of the calibration peptides after calibration of the instrument. With about 60% of measurements, this method is adequate for unambiguous identification. For the remainder it is necessary to fragment the tryptic fragments via MSMS. The peptides of interest are checked according to the hit list to see whether they are suitable for MSMS, i.e. they must have a particular “minimum intensity” and there must be no other signals in the direct vicinity or even overlapping. If these conditions are satisfied, the corresponding ion is fragmented and at least parts of the sequence are obtained. These can be used to check in each case whether the underlying sequence from the hit list is correct (=confirmation of the hit) or not (=hit is very probably eliminated). If the precursor ion had strong intensity, a complete sequence is obtained (=sequence tag), and the protein identification takes place via the sequence tag. The identification can then be regarded as 100% for an AA number of 9 and above. If it is smaller, a second ion should also be fragmented. Fragmenting one ion after another by MSMS not only results in a larger covering of the protein sequence (=>identification of splice variants), but it is also possible for proteins lying “below” the dominant to be unambiguously ident.
   Mass Spectrometry and Subsequent Comparison of the MALDI NS HITS with the Paramecium Ion Channel Sequence

It was possible to find unambiguous homologies of the retina AC bands with Na/K-ATPases in all three bands (FIG. 6-8). In addition, homologies were found with in some cases unknown and still incompletely annotated proteins (FIG. 6-8). To improve analysis of the mass spectrometry hit identifications, the database entries found were subsequently compared by local alignment analysis (DNASTAR, Megalign, Lipman-Pearson algorithm, structure matrix). The results with greatest identity for the individual bands are:

Band 1 (CH1) Best Local Alignments of AC_{—}Paramecium and 50 Hits from MALDI-MS Band 1 (CH1)

1. Genbank VERSION NP_{—}000694.1 GI:4502273: ATPase, Na+/K+ transporting, alpha 3 polypeptide [Homo sapiens] Lipman-Pearson Protein Alignment
2. Genbank VERSION NP_{—}060208.1 GI:8923251: hypothetical protein FLJ20278
3. Genbank VERSION BAB278657.1 GI:12847659: ISS-homolog to RETINOBLASTOMA-ASSOCIATED PROTEIN HEC˜putative [i Mus musculus]
4. Genbank VERSION 1309271B GI:358960: ATPase alpha2,NaK
5. Genbank VERSION BAB21777.2 GI:20521964: KIAA1686 protein [Homo sapiens]
6. Genbank VERSION XP_{—}134201.2 GI:23623442: similar to Splicing factor 3 subunit 1 (Spliceosome associated protein 114) (SAP 114) (SF3a120 [Mus musculus].
7. Genbank VERSION NP_{—}061885.2 GI:19923493: phosphoinositol 3-phosphate-binding protein-2 [Homo sapiens]
8. Genbank VERSION XP_{—}153583.1 GI:20853645: hypothetical protein XP_{—}153563 [Mus musculus]
9. Genbank VERSION XP_{—}156407.1 GI:20891835: similar to Polyadenylate-binding protein 4 (Poly(A) binding protein 4) (PABP 4) (Inducible poly(A)-binding protein) (IPABP)
10. Genbank VERSION HEC protein [Mus musculus]
11. Genbank VERSION AAM44457.1 GI:21262188: CTCL tumor antigen L14-2 [Homo sapiens].
   Band 2 (CH2): Best Local Alignments of AC_{—}Paramecium and 50 Hits from MALDI-MS Band 1 (CH2)
1. Genbank Version BAB15361.1 GI:10438848: unnamed protein product [Homo sapiens].
2. Genbank Version NP_{—}003820.1 GI:4505231: multiple PDZ domain protein [Homo sapiens].
3. Genbank Version AAA51803.1 GI:179212: Na+ K+ ATPase alpha subunit.
   Band 3 (CH3): Best Local Alignments of AC_{—}Paramecium and 50 Hits from MALDI-MS Band 1 (CH3)
1. GENBANK VERSION XP_{—}106022.4 GI:20533458: hypothetical protein XP_{—}160558
2. GENBANK VERSION XP178179.1 GI:23592991: similar to myosin IXA [Rattus noregsicus] [Mus musoulus].
3. GENBANK VERSION Q9WU82 GI:9972860: Beta-catenin
4. GENBANK VERSION BAA20767.1 GI:2224557: KIAA0308 [Homo sapiens].
5. GENBANK VERSION hypothetical protein XP_{—}106022 [Homo sapiens].

1-47. (canceled)

48. A protein comprising an adenylate cyclase domain and an ion channel domain.

49. The protein of claim 48, wherein the ion channel domain is a potassium ion-channel domain.

50. The protein of claim 48, wherein the ion channel domain forms an ion channel which is voltage controllable.

51. The protein of claim 48, wherein the ion channel domain has six transmembrane helices and a pore loop.

52. The protein of claim 51, wherein the pore loop is disposed after the sixth transmembrane helix.

53. The protein of claim 51, wherein the pore loop originates from the intracellular side.

54. The protein of claim 48, wherein it additionally has a protein-protein interaction domain.

55. The protein of claim 54, wherein the protein protein interaction domain is a tetratricopeptide repeat-like (TPR) domain.

56. The protein of claim 54, wherein the protein-protein interaction domain is disposed C-terminally.

57. The protein of claim 48, wherein the ion channel domain is disposed N-terminally.

58. The protein of claim 48, wherein it is encoded at least in part by a nucleotide sequence which is at least 65% identical to a nucleotide sequence as shown in SEQ ID NO 1 or SEQ ID NO 2, or parts thereof.

59. The protein of claim 48, wherein it is encoded at least in part by a nucleotide sequence which is at least 70% identical to a nucleotide sequence as shown in SEQ ID NO 1 or SEQ ID NO 2, or parts thereof.

60. The protein of claim 48, wherein it is encoded at least in part by a nucleotide sequence as shown in SEQ ID NO 1 or parts thereof or SEQ ID NO 2 or parts thereof.

61. A nucleotide sequence or parts thereof, which is at least 65% identical to a nucleotide sequence as shown in SEQ ID NO 1 or SEQ ID NO 2.

62. A nucleotide sequence or parts thereof, which is at least 70% identical to a nucleotide sequence as shown in SEQ ID NO 1 or SEQ ID NO 2.

63. The nucleotide sequence as shown in SEQ ID NO 1 or parts thereof.

64. The nucleotide sequence as shown in SEQ ID NO 2 or parts thereof.

65. The nucleotide sequence of claim 61, wherein it codes for a protein having at least one domain selected from the group consisting of an adenylate cyclase domain and an ion channel domain.

66. The nucleotide sequence of claim 65, wherein the ion channel domain is a potassium ion channel domain.

67. A protein or peptide encoded at least in part by a nucleotide sequence which is at least 65% identical to a nucleotide sequence as shown in SEQ ID NO 1 or SEQ ID NO 2, or parts thereof.

68. A protein or peptide encoded at least in part by a nucleotide sequence which is at least 70% identical to a nucleotide sequence as shown in SEQ ID NO 1 or SEQ ID NO 2, or parts thereof.

69. A protein or peptide encoded at least in part by a nucleotide sequence as shown in SEQ ID NO 1 or parts thereof or SEQ ID NO 2 or parts thereof.

70. The protein or peptide of claim 67, wherein it has at least one domain selected from the group consisting of an adenylate cyclase domain and an ion channel domain.

71. The protein or peptide of claim 70, wherein the ion channel domain is a potassium ion channel domain.

72. The protein or peptide of claim 70, wherein the ion channel domain forms an ion channel which is voltage controllable.

73. The protein or peptide of claim 70, wherein the ion channel domain has six transmembrane helices and a pore loop.

74. The protein or peptide of claim 73, wherein the pore loop is disposed after the sixth transmembrane helix.

75. The protein or peptide of claim 73, wherein the pore loop originates from the intracellular side.

76. The protein or peptide of claim 70, wherein it additionally has a protein-protein interaction domain.

77. The protein or peptide of claim 76, wherein the protein-protein interaction domain is a tetratricopeptide repeat-like (TPR) domain.

78. A method for identifying similar nucleotide sequences by the use of at least one sequence selected from the group consisting of:
a nucleotide sequence which is at least 65% identical to a nucleotide sequence as shown in SEQ ID NO 1 or SEQ ID NO 2, or parts thereof, and
a nucleotide sequence as shown in SEQ ID NO 1 or parts thereof, and
a nucleotide sequence as shown in SEQ ID NO 2 or parts thereof.

79. The method of claim 78, wherein the similar nucleotide sequences are from mammalian cells.

80. The method of claim 78, wherein the identification is undertaken with the aid of at least one method selected from the group consisting of bioinformatic and immunological methods.

81. A nucleotide sequence identified in accordance with the method of claim 78.

82. A peptide or protein encoded by a nucleotide sequence which is identified in accordance with the method of claim 78.

83. A method for developing active substances by the use of a nucleotide sequence as claimed in claim 61 or of a peptide or protein encoded by this sequence.

84. The method of claim 83, wherein the similar nucleotide sequences or parts thereof are expressed.

85. The method of claim 83, wherein the similar nucleotide sequences or parts thereof are heterologously expressed.

86. The method of claim 83, wherein the active substances influence the activity of ion channels.

87. The method of claim 86, wherein the ion channels are potassium ion channels.

88. The method of claim 83, wherein the active substances influence the activity of adenylate cyclases.

89. The method of claim 83, wherein the nucleotide sequence or the parts thereof are derived from Plasmodium spec.

90. The method of claim 89, wherein Plasmodium spec. is Plasmodium falciparum.

91. The method of claim 83, wherein the active substances are intended for the treatment of diseases.

92. The method of claim 91, wherein the diseases are at least one disease selected from the group consisting of malaria, cardiovascular disorders and epilepsy.

93. An active substance for the treatment of diseases developed in accordance with a method as claimed in claim 83.

94. The active substance of claim 93, wherein the diseases are at least one disease selected from the group consisting of malaria, cardiovascular disorders and epilepsy.

95. A method for developing active substances by the use of a nucleotide sequence as claimed in claim 63 or of a peptide or protein encoded by this sequence.

96. A method for developing active substances by the use of a nucleotide sequence as claimed in claim 64 or of a peptide or protein encoded by this sequence.

97. A method for developing active substances by the use of a nucleotide sequence as claimed in a similar nucleotide sequence identified in accordance with the method of claim 78, or of a peptide or protein encoded by this sequence.


SEQUENCE LISTING (6 sequences)

SEQ ID NO: 1 - DNA, 2673 bp, Paramecium tetraurelia
aatgagtgaagctcaagtaaatccggaagtagataactaatctgtaatatcagaagacagcaacaatgaagatgttgacgtgtttggtcaagattaagagattgataagtttcctctcttaacgagtattttggatagtagttggtctcaaatattagtaaatctcttaactatatatgctctatttggcgatgatattagaattattgcctttgataagagggctgatgatggatttgatgtgatcaccataatatgcatgataatattttcaatagaaatcatcattgccagtttggttagaactgactattttaattcattctttttttggcttgatataatatcaacagtttcttagattttggatataacctcgttcaatattgcagttggtttataaggttctgtggctgcaaaatccgcctcttaattgtcccaagcaaataaagcatctaaaacaagttcgaaagcaattcgagtggttcgacttgttagattaattagaatagttaagttgtataaggctgcaagctattcataagaataagcctttaaaagacagcccatccgaacttagactacgaaaaaaagcaaagcaacaatttatccctcggtgtacgaaaatattaacaatgggccagttgaacaggagaatctaaataaaaaggagaataatactgaataaagggaaggagaagataatgcgaatagttaagaaaatcgagaaaattaaaatatggttgcaaaataacaataaattgacaattctattccaaaggtctaataaattcgtagacaatccatgaaagagccatcgtaagtcaaagccaaagaaaaaaaagaatccagagtgagcaaaaggttgtcggattccacaacgaagaaagtcatcatccttgtcattttattactcttaataatgcctttatttagttctgattactattttgaaccatcctatagtttagcatatgcagctgagtatgtaagagtagtggctgagattccaaatacaaaactaactgagatcaattagacaatatatttcgtgattgattagcataaatcatttgacacgccagtaggatacattaccaacccatttactgagatagagaattatgaaaccccttcatattagtatttaagagaaagttcgaaaagctattattttgaattagtagatcctgtattggtgggtttggaatacattggtgatcctgtcattctttttgtctcagataattctgatgttgagtctaagaattcaatcatcaatattgttaatactttatttgtgagtgccgtattgttatttggagcaattgccttttctaatgatgctaataatgttgccttgaaaccaatagataggatgatagtgaaagtcaatttaatagctaagaatccctaattggccaaggagatgaaattagaatctgatggaactcaaaaggaaaccacttagatagaaaatgctatcatcaaaataggggccttgttggcattgggatttggtgatgcaggttctgctattataggaactaatatggcatcatcaggagatgtggatcctatgttaccaggtaaacgtaaatgggcaatttatggattttgtgatattcgtaatttcactgatgccactgaagtcttgcaaaaagatgtgatgttgtttgtaaataatatagctgagattgttcattcaatggtggatcgatatcaaggatcagccaataaaaacataggagatgcattcctattagtttggaaaatcaatgattcttaatggtatgaagatggaaatgaaatcaaatggtccaacttgacattcataaactaattagctgattgtgctctaattgctttcatgaagatatatgctaagatcaatagagaacctaaaatattagaatatagaaatgatgagagattaagtcaacgtttgccaggatataaagtaaagatgggattcggattgcatataggatggggcatagaaggtgccattggatcagaattcaaaattgatgcctcttatctgagtccaaatgttaatatggcttctcgtttagaagctgctactaaacaatatggtgtggctgtcttaatctcaagcgaattgcaccaatatttctccaatgaagtcaagaaatacaccagatagattgataaagtcactgttaagggaagtgtcaaacctattgggttgttcactgtggagatggaggctgatgatctgcctcctagcaaataggattatccttaagaagagaagtaataagtgatgtatgagaaaaagcaaatctttctttaataactagaatctggggatttcaatgcagaattgtatataaaaagaaacaaggatcttggacttataacgaagaatgttaatcaagaattcttgcatcaatttggctaagggttttaaggttatctcttgggaaattggaaagaggcccacatgttgtttgaaaaagcgaagtaaatcagacctaatgatggtcccataagtactctatttaatgtaatgggggaaacaaatttcaaagctccttcagattggaaaggctatagagagttaacagaaaaatgaatattttaagtctataatgttcggcatttcttttgtttcgaaattatatattataagaaaaaaaaaaaaaaaaa

SEQ ID NO: 2 - DNA, 2999 bp, Plasmodium falciparum
atgccagaaccaaaacaaatatatgcaaataatatttttgataatgataagttgaaaagatttttcttaaaatatagatcaaaagaaaaagtattatattcctttacatcagaaaatgacatcataaatgaagagaagagaaaaagcaaaaataacttgtgcatgttaaaatataaaaatatatgtcttaatatacatacaagcaaatttttcaaatactccaaatgggtatatttatttttgatcatattttcaaaagattttttctatcttcttttaaacaaaaaatatgattttatatcagatatttttatttccattctcttatttggatgtcttatcgaaataatttttaattttttaatggattcaacatatatcttttattttatgttttttgatattatatcgttcatgctgttattctttgatatctttcttttcgaaaaatatttttttgacttttttatttactttacaaatagcttatatacatggaaaaaaattgacaaagataatataatatacctaatacaattatttaagtccttgcgaataattaagatatatcgcttcgtaattaattttataaaaaaacatactaaggaaaaatacaagcacagaaatgagtggaattttgaaaaaatggaaagtatgaaaaatagaccattgaaggaaagtttaaagttcacaaataaaatgcacttagctcttatcaaaagatatttcatgtctttaatttttataatgttatcttatataatgatagaaataatatatatctcaaaggagagcaaaagccctatgaattatttcatctataatttggatcttatagtatttgatgaattttatgaaacggaatttttaaaagctctatacttttatagtgctattcagaaaaataaaagagatgaagaatatttaataagtattaaatcaaaaaggaaattgaaaaattttattaacaagaaagaaatagatatatcaggaattaattatctgctatgggattttacaaacttatcacataatgatctattaaaatttgttactccttcaagtgctaaccaaaatgttgaagaagaaattattttggaccatacaattaaaaatataaatgaattgagatactttgaaacaaagatatatgaatccaaagattttatatttcatataaatataaagagatatatagaaagaactattaaaaatattataatattgaaaatatttattataatattttcttttataatattgttctattttacatgtgaattaaatgtcttattatttcctatagaaagcatattgaaaaaattaaaacttatgaaatccaatccgaccttagctttggaaatgcaagaagaattattgaaccatgagttaaaaaatatattaataaacacaaaattgaagaggaaaagcattaaagaaaattatgaaattttaaaaatggaagaaaacttaatgaaattaggaacattaatgttattaggttttggtgaagcaggagcgaaaattatctcaaaaaatattaacgaacaggaaagagtaaatttactgatcaatggagaaatcgtttattctgttttttccttttgcgacataagaaattttacagaaataacagaagttttaaaagaaaaaattatgattttcataaatttgattgctgaaataatacatgagtgttgtgatttctatgggggaacaataaacaaaaatattggtgatgcatttttattagtttggaaatatcaaaaaaaagaatattcgaataagaaaatgaatatgtttaagtctccaaacaataattatgatgaatattcagaaaaagaaaatataaacaggatatgtgatttggcttttctatctacagtacaaacattaattaaactcagaaagtcagaaaaaatacatatattcctaaataatgaaaatatggatgagttaattaagaacaatattctggagttaagttttggacttcattttggatgggctatagaaggagcaataggaagtagttataaaattgatttatcatatttatcagaaaatgtaaatatagctagtcgattacaagatatttctaaaatatacaaaaacaatattgttatatcaggagatttttatgacaatatgtcggagaaatttaaaaattctcttaggaaaattgatagggttactcttaaaggatgtaggaacccattaaatttatatacttttgatatatgtcttaataagattacgaagaaagtcaatatggaaaattttgacgccaagcctcattttgacgtaaaacttttaaaggtttttgatgatataaaaaagaaggccgaacgaaaaaaaagaaaaaaagaagttcttaatctttcatataatctttatgaagaatatgcaaaaaatgacgacataaaattcataaagatacactatcctaaagattatttggaacaattcaaaattgcccttgagtcatatttaatagggaaatggaatgaatcaaaaaatatattagagtatttaaaaagaaataatatttttgaagatgaaattcttaatcagctgtggaactttttgagtatgaacaattttattgcacccagtgattggtgtggatatagaaaatttttacaaaagtcataaaaattatttatatatatataaaatattgatactattaaaaggattgagttaattatttttttttttaaaaacaaaaaaaattgaaataataaataaatataaatataaatatatatatatgtatatatatatatatttaatgttgtacataaaaaatatataaataattacatatgggagttatataaaaaataaattatatgtcaattataatatatatatatatatatatatatattttatattttatttaatttttttataattacatatggataacatttttatgattttcatttttgttattactcatacaagggcgtttaaactttcaacatacttat

SEQ ID NO: 3 - protein, 865 aa, Paramecium tetraurelia
MSEAQVNPEVDNQSVISEDSNNEDVDVFGQDQEIDKFPLLTSILDSSWSQILVNLLTIYALFGDDIRIIAFDKRADDGFDVITIICMIIFSIEIIIASLVRTDYFNSFFFWLDIISTVSQILDITSFNIAVGLQGSVAAKSASQLSQANKASKTSSKAIRVVRLVRLIRIVKLYKAASYSQEQAFKRQPIRTQTTKKSKATIYPSVYENINNGPVEQENLNKKENNTEQREGEDNANSQENRENQNMVAKQQQIDNSIPKVQQIRRQSMKEPSQVKAKEKKESRVSKRLSDSTTKKVIILVILLLLIMPLFSSDYYFEPSYSLAYAAEYVRVVAEIPNTKLTEINQTIYFVIDQHKSFDTPVGYITNPFTEIENYETPSYQYLRESSKSYYFELVDPVLVGLEYIGDPVILFVSDNSDVESKNSIINIVNTLFVSAVLLFGAIAFSNDANNVALKPIDRMIVKVNLIAKNPQLAKEMKLESDGTQKETTQIENAIIKIGALLALGFGDAGSAIIGTNMASSGDVDPMLPGKRKWAIYGFCDIRNFTDATEVLQKDVMLFVNNIAEIVHSMVDRYQGSANKNIGDAFLLVWKINDSQWYEDGNEIKWSNLTFINQLADCALIAFMKIYAKINREPKILEYRNDERLSQRLPGYKVKMGFGLHIGWGIEGAIGSEFKIDASYLSPNVNMASRLEAATKQYGVAVLISSELHQYFSNEVKKYTRQIDKVTVKGSVKPIGLFTVEMEADDLPPSKQDYPQEEKQQVMYEKKQIFLQQLESGDFNAELYIKRNKDLGLITKNVNQEFLHQFGQGFQGYLLGNWKEAHMLFEKAKQIRPNDGPISTLFNVMGETNFKAPSDWKGYRELTEK

SEQ ID NO: 4 - protein, 885 aa, Plasmodium falciparum
MPEPKQIYANNIFDNDKLKRFFLKYRSKEKVLYSFTSENDIINEEKRKSKNNLCMLKYKNICLNIHTSKFFKYSKWVYLFLIIFSKDFFYLLLNKKYDFISDIFISILLFGCLIEIIFNFLMDSTYIFYFMFFDIISFMLLFFDIFLFEKYFFDFFIYFTNSLYTWKKIDKDNIIYLIQLFKSLRIIKIYRFVINFIKKHTKEKYKHRNEWNFEKMESMKNRPLKESLKFTNKMHLALIKRYFMSLIFIMLSYIMIEIIYISKESKSPMNYFIYNLDLIVFDEFYETEFLKALYFYSAIQKNKRDEEYLISIKSKRKLKNFINKKEIDISGINYLLWDFTNLSHNDLLKFVTPSSANQNVEEEIILDHTIKNINELRYFETKIYESKDFIFHINIKRYIERTIKNIIILKIFIIIFSFIILFYFTCELNVLLFPIESILKKLKLMKSNPTLALEMQEELLNHELKNILINTKLKRKSIKENYEILKMEENLMKLGTLMLLGFGEAGAKIISKNINEQERVNLLINGEIVYSVFSFCDIRNFTEITEVLKEKIMIFINLIAEIIHECCDFYGGTINKNIGDAFLLVWKYQKKEYSNKKMNMFKSPNNNYDEYSEKENINRICDLAFLSTVQTLIKLRKSEKIHIFLNNENMDELIKNNILELSFGLHFGWAIEGAIGSSYKIDLSYLSENVNIASRLQDISKIYKNNIVISGDFYDNMSEKFKNSLRKIDRVTLKGCRNPLNLYTFDICLNKITKKVNMENFDAKPHFDVKLLKVFDDIKKKAERKKRKKEVLNLSYNLYEEYAKNDDIKFIKIHYPKDYLEQFKIALESYLIGKWNESKNILEYLKRNNIFEDEILNQLWNFLSMNNFIAPSDWCGYRKFLQKSQ

SEQ ID NO: 5 - DNA, 2618 bp, konstliche Expressionskassette, Paramecium tetraurelia
ggcgccgccaccatgagcgaggcccaggttaaccccgaagtcgataaccagtccgtgatctccgaggactccaacaacgaggacgtggacgtgttcggccaggaccaagagatcgacaagttccccctgctcacctccatcctggactcctcctggtcccaaatcctggtgaacctgctcaccatctacgccctgttcggcgacgatatccgcatcatcgccttcgacaagcgggccgacgacggcttcgacgtgatcaccatcatctgcatgatcatcttctccatcgaaatcatcatcgcctccctggtccgcaccgactacttcaactccttcttcttctggctggacatcatctccaccgtgtcccaaatcctggacatcactagtttcaacatcgccgtcggcctgcagggctccgtggccgcgaagtccgcctcccaactgtcccaagccaacaaggcctccaagacctcctccaaagccatccgcgtggtgcgcctggtgcgcctgatccgcatcgtgaagctgtacaaggccgcctcctactcccaagagcaagctttcaagcgccagcccatccgcacccagaccaccaagaagtccaaggccaccatctacccctccgtgtacgagaacatcaacaacggccccgtggaacaagagaacctgaacaagaaggagaacaacaccgagcagcgcgagggcgaggacaacgccaactcccaagagaaccgcgagaaccagaacatggtcgccaagcagcagcaaatcgataactccatccccaaagtccagcagatccgccgccaatccatgaaggagccctcgcaagtgaaggccaaggagaagaaggagtcccgcgtgtccaagcgcctgtccgactccaccaccaagaaggtcatcatcctggtgatcctgctcctgctcatcatgcccctgttctcctccgactactactttgaaccctcctactccctggcctacgccgccgaatacgtacgcgtggtggccgagatccccaacaccaagctgaccgagatcaaccagaccatctacttcgtcatcgaccagcacaagtccttcgacacccccgtcggctacatcaccaaccccttcaccgagatcgagaactacgagaccccctcctaccaatacctgcgcgagtcctccaagtcctactacttcgagctcgtggaccccgtgctggtcggcctggagtacatcggcgaccccgtcatcctgttcgtgtccgacaactccgacgtcgaatccaagaactccatcatcaacatcgtgaacaccctgttcgtctccgccgtgctgctgttcggggccatcgccttctccaacgacgccaacaacgtggccctgaagccaattgaccgcatgatcgtcaaagtcaacctgatcgccaagaacccccaactggcgaaggagatgaagctggagtccgacggcacgcagaaggagaccacccaaatcgagaacgccatcatcaagatcggcgcactgctggcactcggattcggagatgctggatctgccatcatcggaacgaacatggcatcatctggagacgtcgaccccatgctgcccggcaaacgcaaatgggccatctacggcttctgcgacatccgcaacttcaccgacgccacggaagtcctgcagaaggacgtcatgctgttcgtcaacaacatcgccgagatcgtgcactccatggtcgatcgctaccaaggctccgccaacaagaacatcggcgacgccttcctgctcgtgtggaagatcaacgacagccagtggtacgaggacggcaacgagatcaagtggtccaacctgaccttcatcaaccagctagccgactgcgccctgatcgccttcatgaagatctacgccaagatcaaccgcgagcccaagatcctggaataccgcaacgacgagcgcctgtcccaacgcctccccggctacaaagtcaagatgggcttcggcctgcacatcggctggggcatcgagggggccatcggctccgagttcaagatcgacgcgtcctacctgtcccccaacgtgaacatggcctcccgcctggaagccgccacgaagcagtacggcgtggccgtgctgatcagcagcgagctgcaccagtacttctccaacgaggtcaagaagtacacccgccagatcgacaaagtcaccgtgaagggctccgtgaagcccatcggcctgttcaccgtggagatggaggccgacgacctgccgccttcgaaacaggactacccgcaagaggagaagcagcaagtcatgtacgagaagaagcagatcttcctgcaacaactggagtccggcgacttcaacgccgagctgtacatcaagcgcaacaaggacctgggcctgatcaccaagaacgtgaaccaggagttcctgcaccagtttggccaaggcttccagggctacctgctgggcaactggaaggaggcccacatgctgttcgagaaggccaagcagatccgccccaacgacggccccatctccaccctgttcaacgtcatgggcgagaccaacttcaaggccccgagcgactggaagggctaccgcgagctgaccgagaagtgagcggccgc

SEQ ID NO: 6 - DNA, 2679 bp, konstliche Expressionskassette, Plasmodium falciparum
ggatccgccgccaccatgcccgagcccaagcagatctacgccaacaacatcttcgacaacgacaagctgaagcgcttcttcctgaagtaccgctccaaggagaaggtcctgtactccttcacctccgagaacgacatcatcaacgaggagaagcgcaagtccaagaacaacctgtgcatgctgaagtacaagaacatatgcctgaacatccacacctccaagttcttcaagtactccaagtgggtctacctgttcctgatcatcttctccaaggacttcttctacctgctactgaacaagaagtacgacttcatctcggacatattcatctcgatcctgctgttcggctgcctgatcgaaatcatctttaacttcctgatggactctacgtacatcttctactttatgttcttcgacatcatctcattcatgctgctgttcttcgacatcttcctgttcgagaagtacttcttcgactttttcatctacttcaccaactccctgtacacctggaagaagatcgacaaggacaacatcatctacctgatccaactgttcaagtccctgcgcatcatcaagatctaccgcttcgtcatcaacttcatcaagaagcacaccaaggagaagtacaagcaccgcaacgagtggaacttcgaaaagatggagtccatgaagaaccgccccctgaaggagtccctgaagttcaccaacaagatgcacctggccctgatcaagcgctacttcatgtccctgatcttcatcatgctgtcctacatcatgatcgagatcatatacatctccaaggagtccaagtcgcccatgaactacttcatctacaacctggacctgatcgtgttcgacgagttctacgagaccgagttcctgaaggccctgtacttctactccgccatccaaaagaacaagcgcgacgaggagtacctgatctccatcaagtccaagcgcaagctgaagaacttcatcaacaagaaggagatcgatatatccggcatcaactacctgctgtgggacttcaccaacctgtcccacaacgacctgctgaagttcgtgaccccgtcctccgccaaccaaaacgtggaggaggaaatcatcctggaccacaccatcaagaacatcaacgagctgcgctacttcgagaccaagatctacgagtccaaggacttcatcttccacatcaacatcaagcgctacatcgagcgcaccatcaagaacatcataatcctgaagatattcataatcatcttctcctttatcatactgttctacttcacgtgcgagctcaacgtcctgctgttccccatcgagtccatcctgaagaagctgaagctgatgaagtccaaccccaccctggccctggagatgcaggaggagctgctgaaccacgagctgaagaacatcctgatcaacaccaagcttaagcgcaagtccatcaaggagaactacgagatcctgaagatggaggagaacctgatgaagctgggcaccctgatgctgctgggcttcggcgaggccggggccaagatcatctccaagaacatcaacgagcaggaacgcgtcaacctgctgatcaacggcgagatcgtgtactccgtgttctccttctgcgacatccgcaacttcaccgagatcaccgaggtcctgaaggagaagatcatgatcttcatcaacctgatcgccgagatcatccacgagtgctgcgacttctacggcggcaccatcaacaagaacatcggcgacgccttcctgctggtctggaagtaccagaagaaggagtactccaacaagaagatgaacatgtttaaatcccccaacaataactacgacgagtactccgagaaggagaacatcaaccgcatctgcgacctggccttcctgtccaccgtgcaaaccctgatcaagctgcgcaagtccgagaagatccacatcttcctgaacaacgagaacatggacgagctgatcaagaacaacatcctggagctgtccttcggcctgcacttcggctgggccatcgagggcgcgatcggctcctcctacaagatcgacctgtcctacctgtccgagaacgtcaacatcgcctcccgactgcaagacatctccaagatctacaagaacaacatcgtcatctccggcgacttctacgacaacatgtccgagaagttcaagaactccctgcgcaagatcgaccgcgtcaccctgaagggctgccgcaaccccctgaacctgtacaccttcgatatctgcctgaacaagatcaccaagaaggtcaacatggagaacttcgacgccaagccccacttcgacgtgaagctgctgaaggtgttcgacgacatcaagaaaaaggccgagcgtaagaagcgcaagaaagaggtcctgaacctgtcctacaacctgtacgaggagtacgccaagaacgacgacatcaagttcatcaagatccactaccccaaggactacctggagcagttcaagatcgccctggagtcctacctgatcggcaagtggaacgagtccaagaacatcctggagtacctgaagcgcaacaacatcttcgaggacgagatcctgaaccagctgtggaacttcctgtccatgaacaacttcatcgccccctccgactggtgcggctaccgcaagttcctgcaaaagtcctgagcggccgcc